(19) United States
(12) Patent Application Publication
(10) Pub. No.: US 2024/0307471 A1
(43) Pub. Date: Sep. 19, 2024

(54) METHOD AGAINST CORONAVIRUS INFECTION USING WATER-EXTRACTED PRODUCT OF MELASTOMA MALABATHRICUM ROOT

(71) Applicant: Chang Gung University, Taoyuan City (TW)

(72) Inventors: Pei-Wen Hsieh, Taoyuan City (TW); Tsong-Long Hwang, Taoyuan City (TW); Yu-Ling Huang, Taoyuan City (TW)

(21) Appl. No.: 18/465,109

(22) Filed: Sep. 11, 2023

(30) Foreign Application Priority Data
Mar. 16, 2023 (TW) 112109805

(51) Int. Cl. A61K 36/185; A61P 31/14

(52) U.S. Cl. CPC A61K 36/185; A61P 31/14; A61K 2236/331; A61K 2236/39

(57) ABSTRACT

A method against coronavirus infection includes administering to a subject in need thereof a pharmaceutical composition containing a water-extracted product of Melastoma malabathricum root.

### CROSS-REFERENCE TO RELATED APPLICATION

This application claims priority of Taiwanese Invention Patent Application No. 112109805, filed on Mar. 16, 2023.

### FIELD

The present disclosure relates to a method against coronavirus infection using a water-extracted product of Melastoma malabathricum root.

### BACKGROUND

Coronaviruses are a group of enveloped positive-sense RNA viruses that cause a variety of diseases in animal species including humans, such as severe acute respiratory syndrome coronavirus (SARS-COV) (also known as severe acute respiratory syndrome coronavirus 1 (SARS-COV-1)) and middle east respiratory syndrome coronavirus (MERS-COV). Coronavirus disease 2019 (COVID-19) is an infectious disease caused by severe acute respiratory syndrome coronavirus 2 (SARS-COV-2) which was recently discovered as a new strain of coronavirus. Major symptoms of COVID-19 include respiratory symptoms such as fever above 38° C., cough, shortness of breath, and difficulty in breathing. Symptoms such as loss of smell and taste, diarrhea, headache, chills, loss of appetite, general malaise, and impaired consciousness may be observed. At present, an effective curative treatment for COVID-19 has not been established, and symptomatic treatment is prioritized.

Melastoma malabathricum, known also as Malabar melastome, is a species of Melastoma in the family Melastomataceae. Melastoma malabathricum is an erect, free-flowering shrub that grows in tropical and subtropical environments, mainly in Southeast Asia. The whole plant of Melastoma malabathricum can be used as herbal medicines. Studies have indicated that various parts of Melastoma malabathricum can exhibit a broad spectrum of pharmacological activity, such as anti-inflammatory, antioxidant, antinociceptive, antidiarrheal, and wound healing activities.

It has been reported in S. M. Joffry et al. (2012), Evid Based Complement Alternat. Med., 258434 that a methanol extract of Melastoma malabathricum aerial part can exhibit significant activity against poliovirus and herpes simplex virus 1 (HSV-1), and a methanol extract of Melastoma malabathricum leaves can exhibit significant activity against measles virus and herpes simplex virus 1 (HSV-1).

In spite of the aforesaid, there is still a need to develop a new strategy that is effective against coronavirus infection.

### SUMMARY

Accordingly, an object of the present disclosure is to provide a method against coronavirus infection, which can alleviate at least one of the drawbacks of the prior art, and which includes administering to a subject in need thereof a pharmaceutical composition containing a water-extracted product of Melastoma malabathricum root.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 shows the high performance liquid chromatography (HPLC) spectrum of the water-extracted product of Melastoma malabathricum root.

FIG. 2 shows the IC₅₀ values determined in the test sample W and the test sample M of Example 3, infra, in which the symbol “***” represents p<0.001 (compared with the test sample M).

FIG. 3 shows the viral infection rate determined in each group of Example 4, infra.

### DETAILED DESCRIPTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art, in Taiwan or any other country.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a method against coronavirus infection, which includes administering to a subject in need thereof a pharmaceutical composition containing a water-extracted product of Melastoma malabathricum root.

As used herein, the term “against coronavirus infection” or “anti-coronavirus infection” means prevention of infection by a coronavirus, suppression of coronavirus replication, and/or treatment and/or prevention of infectious diseases caused by a coronavirus.

As used herein, the term “administration” or “administering” means introducing, providing or delivering a pre-determined active ingredient to a subject by any suitable routes to perform its intended function.

As used herein, the term “subject” refers to any animal of interest, such as humans, monkeys, cows, sheep, horses, pigs, goats, dogs, cats, mice, and rats.

According to the present disclosure, the coronavirus infection may be caused by a coronavirus selected from the group consisting of severe acute respiratory syndrome coronavirus 1 (SARS-COV-1), severe acute respiratory syndrome coronavirus 2 (SARS-COV-2), middle east respiratory syndrome coronavirus (MERS-COV), and combinations thereof.

In certain embodiments, the coronavirus infection is caused by SARS-COV-2.

According to the present disclosure, the preparation processes and conditions of the water-extracted product of Melastoma malabathricum root are within the expertise and routine skills of those skilled in the art

In certain embodiments, the water-extracted product of Melastoma malabathricum root may be obtained by subjecting the fresh root of the plant of Melastoma malabathricum to a water extraction treatment.

In certain embodiments, the water-extracted product of Melastoma malabathricum root may be obtained by subjecting a processed root of Melastoma malabathricum to a water extraction treatment.

In certain embodiments, the processed root of Melastoma malabathricum may be obtained by subjecting the fresh root of the plant of Melastoma malabathricum to a preliminary treatment selected from the group consisting of a drying treatment, a grinding treatment, a chopping treatment, a comminuting treatment, and combinations thereof.

In certain embodiments, the water extraction treatment may be performed by using the root of Melastoma malabathricum and water in a weight ratio ranging from 1:1 to 1:20. In an exemplary embodiment, the weight ratio of the root of Melastoma malabathricum to water is 1:10.

In certain embodiments, the water extraction treatment may be performed at a temperature ranging from 60° C. to 150° C. for a time period ranging from 0.5 hour to 4 hours. In an exemplary embodiment, the water extraction treatment is performed at a temperature of 100° C. for 2 hours.

According to the present disclosure, the water-extracted product of Melastoma malabathricum root may be subjected to a membrane filtration treatment using techniques well-known to those skilled in the art.

In certain embodiments, the membrane filtration treatment may be conducted using three membranes having molecular weight cut-off values (MWCO values) of 10 kilo Dalton (kDa), 100 kD, and 300 kDa, respectively, so as to obtain four fractions having molecular weights of lower than 10 kDa, from 10 kDa to 100 kDa, from 100 kDa to 300 kDa, and greater than 300 kDa, respectively.

According to the present disclosure, the pharmaceutical composition may be formulated into a dosage form suitable for oral administration, parenteral administration, topical administration, or respiratory tract administration using technology well known to those skilled in the art.

According to the present disclosure, the dosage form suitable for oral administration includes, but is not limited to, sterile powders, tablets, troches, lozenges, pellets, capsules, dispersible powders or granules, solutions, suspensions, emulsions, syrup, elixir, slurry, and the like.

For parenteral administration, the pharmaceutical composition according to the present disclosure may be formulated into an injection, e.g., a sterile aqueous solution or a dispersion.

The pharmaceutical composition according to the present disclosure may be administered via one of the following parenteral routes: intraperitoneal injection, intrapleural injection, intramuscular injection, intravenous injection, intraarterial injection, intraarticular injection, intrasynovial injection, intrathecal injection, intracranial injection, intraepidermal injection, subcutaneous injection, intradermal injection, intralesional injection, and sublingual administration.

According to the present disclosure, the pharmaceutical composition may be formulated into an external preparation suitable for topical application to the skin using technology well known to those skilled in the art. The external preparation includes, but is not limited to, emulsions, gels, ointments, creams, patches, liniments, powder, aerosols, sprays, lotions, serums, pastes, foams, drops, suspensions, salves, and bandages.

According to the present disclosure, the pharmaceutical composition may be formulated into a spray (e.g., a nasal spray or an oral spray) suitable for oral inhalation or nasal inhalation.

According to the present disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, and the like. The choice and amount of the aforesaid agents are within the expertise and routine skills of those skilled in the art.

The dose and frequency of administration of the pharmaceutical composition of the present disclosure may vary depending on the following factors: the severity of the illness or disorder to be treated, routes of administration, and age, physical condition and response of the subject to be treated. In general, the pharmaceutical composition may be administered in a single dose or in several doses.

The disclosure will be further described by way of the following examples. However, it should be understood that the following examples are solely intended for the purpose of illustration and should not be construed as limiting the disclosure in practice.

### Examples

### General Experimental Materials:

### 1. Source and Cultivation of Human Angiotensin-Converting Enzyme 2 (ACE2) Stable Expressing Cell Line HEK293T

Human ACE2 stable expressing cell line HEK293T (abbreviated as hACE2-HEK293T cells) was purchased from the RNA Technology Platform and Gene Manipulation Core, Academia Sinica, Taipei, Taiwan. The hACE2-HEK293T cells were grown in Dulbecco's Modified Eagle's Medium (DMEM) (Gibco) supplemented with 10% fetal bovine serum (FBS) (Gibco), 10 μg/mL blasticidin (Sigma), 100 U/mL penicillin (Gibco), and 0.1 mg/ml streptomycin (Gibco), and were cultivated in an incubator with culture conditions set at 37° C. and 5% CO₂. Medium change was performed every two to three days. Cell passage was performed when the cultured cells reached 80% to 90% of confluence.

### General Procedures:

### 1. Statistical Analysis

All the experiments described below were performed in triplicates. The experimental data of all the test groups are expressed as mean±standard error of the mean (SEM), and were analyzed using Student's t-test, so as to evaluate the differences between the groups. Statistical significance is indicated by p<0.05.

### Example 1. Preparation of Water-Extracted Product of Melastoma malabathricum Root

A lyophilized powder of Melastoma malabathricum root (which was purchased from Weei Ywun International Herb Co., Ltd.) and water were mixed in a weight ratio of 1:10, followed by conducting heat reflux extraction at 100° C. for 2 hours. The resultant mixture was subsequently filtered using a filter with a porosity of 6 μm (ADVANTEC), so as to obtain a first filtrate. The residue was subjected to further extraction by repeating the above-mentioned treatments, so as to obtain a second filtrate. The first and second filtrates were collected and served as a water-extracted product of Melastoma malabathricum root.

The water-extracted product of Melastoma malabathricum root was subjected to a drying treatment using a rotary evaporator and a centrifugal evaporator to remove water, so as to obtain a dried powder of the water-extracted product of Melastoma malabathricum root.

### Example 2. High Performance Liquid Chromatography (HPLC) Analysis

### Experimental Procedures:

The dried powder of the water-extracted product of Melastoma malabathricum root obtained in Example 1 was dissolved in an appropriate amount of 10% acetonitrile, thereby obtaining a test sample of the water-extracted product of Melastoma malabathricum root having a concentration of 1 mg/mL.

The test sample was subjected to high performance liquid chromatography (HPLC) analysis using technology well-known to those skilled in the art. The operating parameters and conditions for performing HPLC are summarized in Table 1 below.

TABLE 1

| | Prominence HPLC system | |
|---|---|---|
| | (Shimadzu, LC-20AT) | |
| | equipped with a UV/Vis detector | |
| HPLC instrument | (Jasco, UV-1575 UV) | |
| Type of chromatography | C18 Column (Supelco, Ascentis ®) | |
| column | | |
| Size of chromatography | Length: 250 mm; | |
| column | inner diameter: 4.6 mm | |
| Temperature of | 25° | C. |
| chromatography column | | |
| Sample injection volume | 20 | μL |
| Detection wavelength | 275 | nm |
| Mobile phase | 10% acetonitrile/0.05% trifluoroacetic acid | |
| Flow rate of test sample | 1 | mL/min |

### Results:

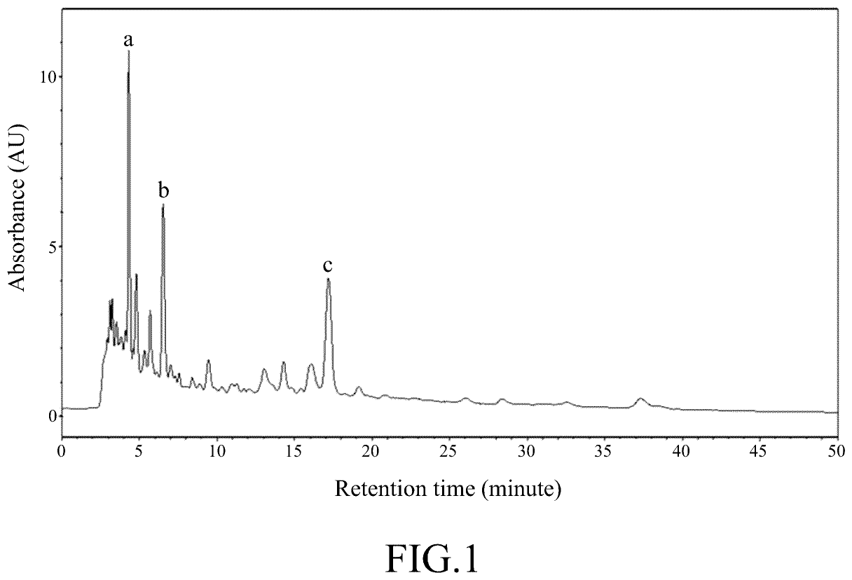

FIG. 1 shows the HPLC spectrum of the water-extracted product of Melastoma malabathricum root. As shown in FIG. 1, there were three main peaks (i.e., peaks a, b, and c) during a 50-minute retention period, indicating that there were three major components in the water-extracted product of Melastoma malabathricum root.

### Example 3. Evaluation for the Effect of Water-Extracted Product of Melastoma malabathricum Root Against Severe Acute Respiratory Syndrome Coronavirus 2 (SARS-COV-2)

### Experimental Procedures:

A. Preparation of Methanol-Extracted Product of Melastoma malabathricum Root

The dried powder of the methanol-extracted product of Melastoma malabathricum root was prepared for comparison purpose using a procedure that is substantially similar to that of the water-extracted product of Melastoma malabathricum root as described in Example 1, except that water was replaced with 100% methanol.

### B. Preparation of Test Solutions

A suitable amount of the dried powder of the water-extracted product of Melastoma malabathricum root obtained in Example 1 was dissolved in deionized water, so as to obtain a stock solution of the water-extracted product of Melastoma malabathricum root having a concentration of 4 mg/ml (abbreviated as a stock solution W).

In addition, a suitable amount of the dried powder of the methanol-extracted product of Melastoma malabathricum root obtained in section A of this example was dissolved in 100% methanol, so as to obtain a stock solution of the methanol-extracted product of Melastoma malabathricum root having a concentration of 4 mg/ml (abbreviated as a stock solution M).

A suitable amount of a respective one of the stock solutions W and M was dissolved in deionized water, so as to obtain four test samples having different concentrations (i.e., 160 μg/mL and 400 μg/mL) as shown in Table 2 below.

TABLE 2

| Test sample | Concentration (μg/mL) |
|---|---|
| Test sample W1 | 160 |
| Test sample W2 | 400 |
| Test sample M1 | 160 |
| Test sample M2 | 400 |

### C. Determination of Half-Maximal Inhibitory Concentration (IC₅₀)

A respective one of the four test samples obtained in section B of this example was subjected to determination of inhibition rate using AlphaScreen® SARS-COV-2 Spike-ACE2 Binding Assay Kit (Perkin Elmer). Briefly, 10 μL of the respective test sample and deionized water (serving as a blank control) was added into a respective well of a 96-well culture plate, and was then added with 5 μl of a 146.89 nM human ACE2 protein (His Tag) solution (Sino Biological Inc., Cat. No. 10108-H08H), followed by cultivation in an incubator (37° C.) for 15 minutes. Next, 5 μL of a 121.36 nM SARS-COV-2 Spike RBD-mFc recombinant protein solution (Sino Biological Inc., Cat. No. 40592-V05H) was added into each well, followed by cultivation in an incubator (37° C.) for 1 hour. Afterwards, 10 μL of protein A acceptor beads (20 mg/mL) (Cat. No. AL101C, Perkin Elmer) was added into each well, followed by cultivation in the dark at 25° C. for 1 hour. Thereafter, 10 μL of nickel chelate donor beads (20 mg/mL) (Cat. No. AS101D, Perkin Elmer) was added into each well, followed by cultivation in the dark at 25° C. for 1 hour.

The respective resultant mixture in each well was subjected to determination of luminescence intensity at an excitation wavelength of 680 nm and an emission wavelength of 615 nm using an EnSpire Alpha® multimode plate reader (Model No.: 2390-0000; Perkin Elmer).

The inhibition rate (%) of the respective test sample on the binding of SARS-COV-2 spike protein and ACE2 was calculated using the following Equation (I):

$$A = [ ( B - C ) / B ] \times 100 \qquad ( I )$$

where A=inhibition rate (%)
   B=luminescence intensity determined for blank control
   C=luminescence intensity determined for respective test sample

In addition, the half-maximal inhibitory concentration (IC₅₀) was determined from the linear portion of the plotted dose-response curve by calculating the concentration of the respective test sample that reduced inhibition rate, as compared to the blank control, by 50%.

The data thus obtained were analyzed according to the method described in section 1 of the “General Procedures”.

### Results:

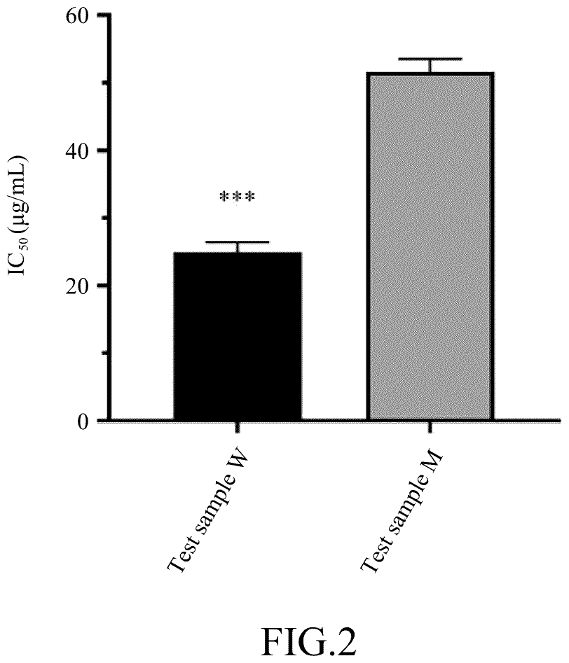

Referring to FIG. 2, the IC₅₀ value determined for the test sample W (i.e., the water-extracted product of Melastoma malabathricum root) was significantly lower than that determined for the test sample M (i.e., the methanol-extracted product of Melastoma malabathricum root).

The result indicates that the water-extracted product of Melastoma malabathricum root is effective in inhibiting the binding of SARS-COV-2 spike protein and ACE2, and hence has a high potential against coronavirus (such as SARS-COV-2) infection.

### Example 4. Evaluation for the Effect of Different Fractions Obtained from Water-Extracted Product of Melastoma malabathricum Root Against SARS-COV-2

A. Preparation of Different Fractions from Water-Extracted Product of Melastoma malabathricum Root

The water-extracted product of Melastoma malabathricum root obtained in Example 1 was subjected to a membrane filtration treatment using a flat-bed filter (TEFLUX, Model No.: TX-PFR-1209-POM-FS) and membranes with three different molecular weight cut-off values (MWCO values) (i.e., 300 kDa, 100 kDa, and 10 kDa in sequence), so as to obtain four fractions having different molecular weights as shown in Table 3 below.

The respective one of the four fractions was subjected to a drying treatment using a rotary evaporator, so as to obtain a dried powder of the respective fraction.

TABLE 3

| Fraction | Molecular weight (kDa) |
|---|---|
| Fraction I | <10 |
| Fraction II | 10-100 |
| Fraction III | 100-300 |
| Fraction IV | >300 |

### B. Pseudovirus Solution

Two pseudovirus solutions, i.e., a solution of VSV-G pseudotyped lentivirus and a solution of SARS-COV-2 spike pseudotyped lentivirus expressing SARS-COV-2 B.1.617.2 (delta strain) spike protein, used in the following experiments were purchased from the RNA Technology Platform and Gene Manipulation Core, Academia Sinica, Taipei, Taiwan.

### C. Determination of Half-Maximal Inhibitory Concentration (IC₅₀)

A respective one of the four dried powders obtained in section A of this example was subjected to determination of half-maximal inhibitory concentration (IC₅₀) according to the procedure described in section C of Example 3.

As shown in Table 4, the IC₅₀ values determined for the fractions II and III were respectively lower than those determined for the fractions I and IV, indicating that the fraction II having a molecular weight ranging from 10 kDa to 100 kDa and the fraction III having a molecular weight ranging from 100 kDa to 300 kDa are effective in inhibiting the binding of SARS-COV-2 spike protein and ACE2, and hence have a high potential to effectively combat coronavirus (such as SARS-CoV-2) infection.

TABLE 4

| Fraction | IC₅₀ value (μg/mL) |
|---|---|
| Fraction I | 51.56 ± 6.61 |
| Fraction II | 21.54 ± 1.39 |
| Fraction III | 19.82 ± 0.69 |
| Fraction IV | 23.18 ± 1.70 |

### D. Preparation of Virus Mixtures

A suitable amount of a respective one of the dried powder of the fraction II and the dried powder of the fraction III was mixed with DMEM, the respective resultant stock solution was then mixed with 69.2 μL of the SARS-COV-2 spike pseudotyped lentivirus solution (with a virus amount of 289 RIU/μL) and 430.8 μL of DMEM, so as to obtain four virus mixtures having different concentrations of fraction (i.e., 125 μg/mL and 375 μg/mL) as shown in Table 5 below.

TABLE 5

| | | Concentration of | Virus amount |
|---|---|---|---|
| Virus mixture | Fraction | fraction (μg/mL) | (RIU/mL) |
| Virus mixture 1 | Fraction II | 125 | 19999 |
| Virus mixture 2 | | 375 | |
| Virus mixture 3 | Fraction III | 125 | |
| Virus mixture 4 | | 375 | |

### E. SARS-COV-2 Pseudovirus Infection Assay

The hACE2-HEK293T cells prepared in section 1 of “General Experimental Materials” were divided into 6 groups, including one blank control group, one pathological control group, and four experimental groups (i.e., experimental groups 1 to 4). Each group of the hACE2-HEK293T cells was incubated in a respective well of a 96-well culture plate containing 100 μL of DMEM (supplemented with 10% FBS, 100 U/mL penicillin, and 0.1 mg/mL streptomycin) at 2×10⁴ cells/well, followed by cultivation in an incubator (37° C., 5% CO₂) for 16 hours.

Afterward, the culture medium in each well was removed, and the hACE2-HEK293T cells of the experimental groups 1 to 4 were respectively infected with the virus mixtures 1 to 4 prepared in section D of this example at a multiplicity of infection (m.o.i.) of 0.1. In addition, the hACE2-HEK293T cells of the pathological control group were treated with DMEM containing a suitable amount of a SARS-COV-2 spike pseudotyped lentivirus solution (with a final virus amount of 19,999 pfu/mL), and the hACE2-HEK293T cells of the blank control group were treated with DMEM containing a suitable amount of a VSV-G pseudotyped lentivirus solution (with a final virus amount of 20,024 pfu/mL).

After cultivation in an incubator (37° C., 5% CO₂) for 24 hours, each of the cell cultures was added with 50 μL of a luminescent substrate (Promega, Cat. No. E2510), followed by cultivation in an incubator (37° C.) for 5 minutes. Next, each group was subjected to determination of luminescence intensity by using a plate reader (Tecan, Model No.: Infinite® 200 PRO).

The viral infection rate (%) was calculated using the following Equation (II):

$$D = [ ( E - G ) / ( F - G ) ] \times 100 \qquad ( II )$$

where D=viral infection rate (%)
   E=luminescence intensity determined in the respective group
   F=luminescence intensity determined in the pathological control group
   G=luminescence intensity determined in the blank control group

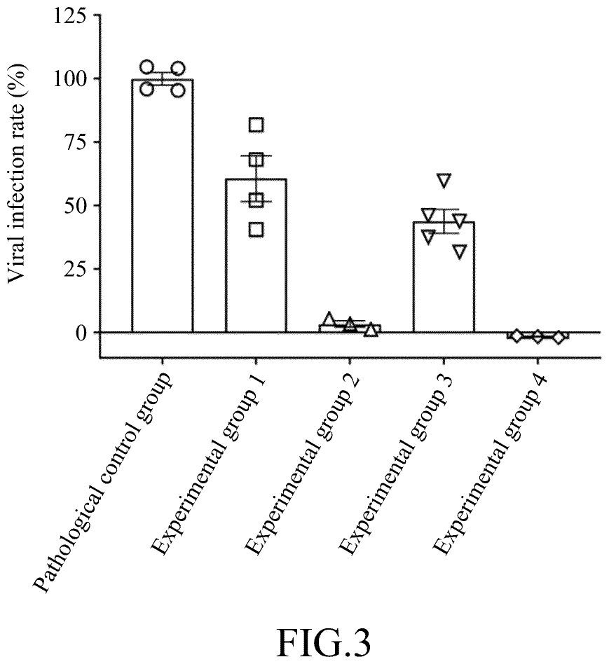

Referring to FIG. 3, the viral infection rates determined in the experimental groups 1 to 4 were respectively lower than that determined in the pathological control group. To be specific, the viral infection rates determined in the experimental groups 2 and 4 were close to zero. These results indicate that the fractions II and III obtained from the water-extracted product of Melastoma malabathricum root can exhibit dose-related antiviral effect.

Summarizing the test results above, it is clear that the water-extracted product of Melastoma malabathricum root and the fractions prepared therefrom can act effectively against coronavirus (such as SARS-COV-2) infection.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method against coronavirus infection, comprising administering to a subject in need thereof a pharmaceutical composition containing a water-extracted product of Melastoma malabathricum root.

2. The method as claimed in claim 1, wherein the coronavirus infection is caused by a coronavirus selected from the group consisting of severe acute respiratory syndrome coronavirus 1 (SARS-COV-1), severe acute respiratory syndrome coronavirus 2 (SARS-COV-2), middle east respiratory syndrome coronavirus (MERS-COV), and combinations thereof.

3. The method as claimed in claim 2, wherein the coronavirus infection is caused by SARS-COV-2.

4. The method as claimed in claim 1, wherein the pharmaceutical composition is in a dosage form selected from the group consisting of a parenteral dosage form, an oral dosage form, a topical dosage form, and an inhalation dosage form.
